(19) United States
(12) Patent Application Publication
(10) Pub. No.: US 2015/0231077 A1
(43) Pub. Date: Aug. 20, 2015

(54) AMINE PASSIVATED NANOPARTICLES FOR CANCER TREATMENT AND IMAGING

(71) Applicant: THE CLEVELAND CLINIC FOUNDATION, Cleveland, OH (US)

(72) Inventors: Shunji Egusa, Cleveland Hts., OH (US); Yogen Saunthararajah, Cleveland, OH (US)

(21) Appl. No.: 14/623,872

(22) Filed: Feb. 17, 2015

Related U.S. Application Data
(60) Provisional application No. 61/940,540, filed on Feb. 17, 2014.

(51) Int. Cl. A61K 9/16; A61K 31/704; A61K 31/519

(52) U.S. Cl. CPC A61K 9/167; A61K 31/519; A61K 31/704

(57) ABSTRACT

Amine-passivated gold nanoparticles and methods of making and using such nanoparticles are described. The nanoparticles can be prepared in a manner in which amine-containing drugs or imaging agents associate with the surface of the nanoparticles to allow delivery of the drugs or agents in vivo, but the association is weak enough to allow the amine-containing drug or imaging agents to be released from the nanoparticle upon reaching its target. Amine passivated gold nanoparticles including targeting molecules which are attached through a thiol linkage can also be prepared and used.

### CROSS-REFERENCE TO RELATED APPLICATIONS

This application claims priority from U.S. Provisional Application Ser. No. 61/940,540, filed Feb. 17, 2014, the entire contents of which are incorporated herein by reference.

### GOVERNMENT FUNDING

This invention was made with government support under Grant Nos. 1R01CA138858 and U54HL090513, awarded by the National Institutes of Health, and Grant No. PR081404, awarded by the Department of Defense. The government has certain rights in the invention.

### BACKGROUND

An enduring, fundamental issue in cancer medicine is that of poor therapeutic index: treatments typically destroy normal as well as cancer cells, causing substantial toxicity that limits the safety and efficacy of treatment. Thus there is a need for methods to more selectively deliver drugs to cancer cells and thereby spare normal cells. Drug delivery technologies such as micellar and liposomal (Peters et al., Proc Natl Acad Sci; 106:9815-9 (2009); Torchilin V P., Nat Rev Drug Discov; 4:145-60 (2005)) or polymeric encapsulations (Kabanov A V, Vinogradov S V., Angew Chem Int Ed Engl; 48:5418-29 (2009); Mitragotri S, Lahann J., Adv Mater; 24:3717-23 (2012)) and monoclonal antibody conjugation (Senter P D., Curr Opin Chem Biol; 13:235-44 (2009)) have been intensively pursued as candidates to improve cancer therapeutics.

Gold (Au) nanoparticles have also been studied for drug delivery. Rana et al., Adv Drug Deliv Rev; 64:200-16 (2012); Akhter et al., Expert Opin Drug Deliv; 9:1225-43 (2012); Arvizo et al., Expert Opin Drug Deliv; 7:753-63 (2010); Boisselier E, Astruc D., Chem Soc Rev; 38:1759-82 (2009); Huang et al., Nanomedicine; 2:681-93 (2007). An important aspect of Au nanoparticles is that their physical properties as well as interactions with bio-organisms can be controlled by their size and shape, and the careful engineering of these combined effects have culminated in various theranostic applications. El-Sayed et al., Cancer Lett; 239:129-35 (2006); Chithrani et al., Nano Lett; 6:662-8 (2006); Egusa et al., J Phys Chem C., 111:17993-6 (2007). Au nanoparticles are typically passivated with thiol-containing molecules via strong thiol-to-Au bond (Feldheim D L, Foss C A. Metal Nanoparticles. New York: Marcel Dekker; 2002; El-Sayed M A., Acc Chem Res; 34:257-64 (2001); Templeton et al., Acc Chem Res; 33:27-36 (2000)), and some of the delivery mechanisms by the thiol-passivated Au nanoparticles have been elucidated. These include (i) accumulation based on EPR (enhanced permeability and retention) effect observed for relatively large-sized nanoparticles (˜15-100 nm) in solid tumor models (Matsumura Y, Maeda H., Cancer Res; 46:6387-92 (1986)), and (ii) various payload release mechanisms including e.g. pH change or triggering by endogenous glutathione utilizing thiol-to-thiol ligand exchange. Ulbrich K, Subr V., Adv Drug Deliv Rev; 56:1023-50 (2004); Hong et al., J Am Chem Soc; 128:1078-9 (2006).

However, existing drug delivery technologies are limited by several problems. Antibody-drug conjugates are very expensive, difficult to synthesize, and generally can only bear 10 or fewer drug molecules. Liposome polymer encapsulation suffer from size non-uniformity, which has negative effects on cellular uptake and efflux, and bio-distribution. Conventional gold nanoparticles which are passivated using covalent and non-reversible thiol-gold association require complicated chemistry of their ligands, both for drug loading and for attaching targeting molecules. Accordingly, there remains a need for a technology for the delivery of compounds in vivo that is relatively simple and inexpensive, while making use of small particles having a relatively predictable size.

### SUMMARY

The inventors have described a simple and versatile synthesis of water-soluble gold nanoparticles passivated with amine-containing molecules, which allow for controlled drug release via ligand exchange with bio-available glutathione. Taking methotrexate-passivated gold nanoparticles (Au:MTX) as an example, drug delivery and controlled release via glutathione-mediated ligand exchange was evaluated. Furthermore, the possibility of using Au:MTX to improve therapeutic index in acute myeloid leukemia (AML) models was examined in vitro and in vivo. Au:MTX exhibited cancer selectivity in vitro. Au:MTX had an elevated potency towards an AML cell line THP-1 in a range of dosage (1-5 nM), and therefore an enhanced delivery of drug, whereas normal hematopoietic stem/progenitor cell (HSPC) growth was minimally affected by Au:MTX and MTX treatments within the same range of dosage. In vivo efficacy and safety of Au:MTX was evaluated in a murine xenotransplant model of primary human AML. Au:MTX treatment, compared to control groups including MTX-only and Au nanoparticle-only treatments, produced better leukemia suppression without added toxicity, indicating an enhanced therapeutic index.

In one aspect, the present invention provides an amine-passivated gold nanoparticle, comprising a gold nanoparticle passivated with a plurality of amine-containing drugs or imaging agents. In some embodiments, the nanoparticle has a diameter from 1 to 5 nanometers, while in further embodiments, the amine-containing drugs or imaging agents have a molecular weight from about 300 to about 1,000 daltons. In yet further embodiments, the amine-passivated gold nanoparticle further comprises a targeting molecule bonded to the nanoparticle through a thiol linkage.

Another aspect of the invention provides a method of making an amine-passivated gold nanoparticle, comprising combining an amine-containing drug or imaging agent with HAuCl₄ and a reducing agent in a polar organic solvent at a temperature from −80 to 20° C. for a time sufficient to form amine-passivated gold nanoparticles. In some embodiments, the gold nanoparticle has a diameter from 1 to 5 nanometers. In another embodiment, the method includes reacting the amine-passivated gold nanoparticle with a thiol-containing targeting molecule.

A further aspect of the invention provides a method of treating cancer in a subject identified as having cancer by administering to the subject a therapeutically effective amount of an amine-passivated gold nanoparticle comprising a gold nanoparticle passivated with an amine-containing anticancer agent. In some embodiments, the amine-passivated gold nanoparticle has a diameter from 1 to 5 nanometers. In other embodiments, the amine-containing compounds have a molecular weight from about 300 to about 1,000 daltons. In a further embodiment, the amine-passivated nanoparticle is administered together with a pharmaceutically acceptable carrier. In a yet further embodiment, the amine-passivated nanoparticle further comprises a targeting molecule bonded to the nanoparticle through a thiol linkage.

### BRIEF DESCRIPTION OF THE FIGURES

The present invention may be more readily understood by reference to the following drawings.

FIG. 1 (a-d) provides graphs and images showing methotrexate (MTX)-passivated Au nanoparticles (Au:MTX). (a) Transmission electron microscope (TEM) image of Au:MTX nanoparticles. (b) Size distribution of Au:MTX (2.6±0.7 nm). Histogram was constructed from multiple TEM images. (c) Schematic drawing of drug loading per nanoparticle. R is the radius of nanoparticle core, and pL is the radius of the drug's footprint (or projected area on the nanoparticle core). With Au atomic radius of ˜1.44 Å and 2R ˜2.6 nm, number of MTX per nanoparticle is ˜75, and footprint is ˜0.28 nm2 with 2 pL ˜6 Å. (d)¹H-NMR of Au:MTX (top panel) and MTX in D₂O (bottom panel).

FIG. 2 (a & b) provides graphs and images showing controlled payload release from Au:MTX nanoparticles via ligand exchange. (a) Glutathione (GSH)-induced MTX release from Au:MTX characterized using UV-vis absorption spectroscopy. GSH concentration was varied as Au-to-GSH molar ratio=1:0.5 (labeled as ×0.5 in the inset), 1:1 (×1), and 1:5 (×5). (b) TEM image of Au nanoparticles after the ligand exchange (Au-to-GSH molar ratio=1:1).

FIG. 3 (a-c) provides graphs and images showing stability of Au:MTX under physiologically relevant conditions. (a) Au:MTX nanoparticles are stable at pH 4-9, and precipitates at pH 2-3. (b) Absorption spectrum of the supernatant of precipitated Au:MTX solution (10 μM equimolar in MTX) at pH=3. MTX molecules are almost fully dissociated from nanoparticles. (c) Au:MTX nanoparticles are stable in saline (PBS) as well as in protein (BSA and FBS) solutions.

FIG. 4 (a & b) provides graphs and images showing in vitro evaluation of drug delivery by Au:MTX nanoparticles. (a) Growth curves of MTX-, Au:MTX-, and Au:FOL-treated THP-1 (an AML cell line). Cells were treated immediately before the incubation started, with PBS, MTX (500 nM, top panel; 50 nM, bottom panel), as well as equimolar Au:MTX and Au:FOL, so that the MTX payload is equivalent to the MTX-only treatment. Au:FOL does not significantly affect the growth of THP-1. (b) Au:MTX uptake by THP-1 cells examined by TEM. Locations of Au nanoparticles are visualized as black dots in the images, using silver-enhancement technique.

FIG. 5 provides graphs showing Au:MTX demonstrating enhanced therapeutic index compared to MTX-alone in vitro. Growth curves of MTX- and Au:MTX-treated (1 nM, left panel; 2 nM middle panel; 5 nM, right panel) cancer cells (THP-1, top panels) and normal hematopoietic stem/progenitor cells (HSPCs, bottom panels) are shown. Au:MTX completely inhibits THP-1 growth at 1-5 nM, whereas MTX-alone show dose-dependent THP-1 growth suppression. Moreover, both Au:MTX and MTX-alone does not significantly affect normal HSPC growth at 1-5 nM.

FIG. 6 (a-f) provides graphs and images showing Au:MTX treatment in a murine xenotransplant model of primary human AML. Therapeutic index of Au:MTX is compared to PBS, MTX, and Au:FOL in vivo. (a) Human AML content in bone marrow measured by flow cytometry. Au:MTX-treated group exhibit markedly improved efficacy. (b) Representative raw flow cytometry data. (c) Murine bones before marrow extraction. Murine bones from Au:MTX-treated group exhibit marked suppression of anemia compared to other three treatment groups. (d) Au nanoparticle delivery in vivo is demonstrated in spleens; and (e) in livers harvested post-treatment with silver enhancement, and eosin staining. Dots in the spleen and liver tissues are the locations of Au nanoparticles visualized via silver-enhancement. (f) Histology of intestines indicate no added damage to endothelium caused by Au:MTX. Scale bars indicate 100 μm in (d, e, f).

FIG. 7 (A-C) provides a schematic, graph, and images showing lineage-specific drug delivery and tracking, proof of concept. (A) EMP/daunorubicin(DNR) Au nano-linkers 1: target EPO-R and internalized in erythroid progenitor; 2: release DNR via ligand exchange with intracellular GSH; 3: Au-core quenched DNR fluorescence, which is recovered upon DNR release. Multi-color flow cytometry elucidated: (B) time-course of EPO-R targeting and drug release via pStat5 activation and DNR fluorescence; and (C) more selective DNR delivery to erythroid progenitors (CD71+) over other cell populations in whole mouse bone marrow, compared to DNR alone.

FIG. 8 provides graphs and images showing the structural characterization of Au nano-linker for targeted delivery. EPO/doxorubicin (DOX) Au nano-linker synthesized via partial ligand exchange (TEM images, left), and NMR spectra confirming the successful synthesis (right).

FIG. 9 provides a graph showing lineage-targeted drug delivery in vitro. EPO-R expressing cell lines (UT7/EPO, TF-1, K562) and GCSF-R expressing cell lines (THP-1, U266, HL-60) were treated with DNR-loaded Au nano-linker with erythroid progenitor-targeting peptide (ETP) or myeloid targeting peptide (MTP). Flow cytometry evaluation of intracellularly released DNR demonstrated erythroid lineage selective DNR delivery by Au:DNR-ETP and myeloid lineage selective delivery by Au:DNR-MTP, respectively.

FIG. 10 provides graphs and images showing lineage-targeted drug delivery in vivo, proof of principle. Normal mouse (n=5 per group) were treated with MTX-loaded Au nano-linker with or without erythroid progenitor-targeting peptide. Flow cytometry evaluation of erythroid progenitor populations in harvested bone marrows clearly demonstrated lineage-targeted delivery of drug (p=0.007).

### DETAILED DESCRIPTION

Amine-passivated gold nanoparticles and methods of making and using such nanoparticles are described. The nanoparticles can be prepared in a manner in which amine compounds associate with the surface of the nanoparticles to allow delivery of the amino compounds in vivo, while the association is weak enough to allow the amine compound to be released from the nanoparticle upon reaching its target. Amine passivated gold nanoparticles including targeting molecules which are attached through a thiol linkage can also be prepared and used.

### DEFINITIONS

It is to be understood that this invention is not limited to particular methods, reagents, compounds, compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural references unless the content clearly dictates otherwise. Thus, for example, reference to “a cell” includes a combination of two or more cells, and the like.

The term “about” as used herein when referring to a measurable value such as an amount, a temporal duration, and the like, is meant to encompass variations of ±20% or 110%, more preferably ±5%, even more preferably ±1%, and still more preferably ±0.1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

“Image” or “imaging” refers to a procedure that produces a picture of an area of the body, for example, organs, bones, tissues, or blood.

“Treat”, “treating”, and “treatment”, etc., as used herein, refer to any action providing a benefit to a subject afflicted with a condition or disease such as cancer, including improvement in the condition through lessening or suppression of at least one symptom, delay in progression of the disease, etc.

A “subject,” as used herein, can be any animal, and may also be referred to as the patient. Preferably the subject is a vertebrate animal, and more preferably the subject is a mammal, such as a domesticated farm animal (e.g., cow, horse, pig) or pet (e.g., dog, cat). In some embodiments, the subject is a human.

“Pharmaceutically acceptable” as used herein means that the compound or composition is suitable for administration to a subject for the methods described herein, without unduly deleterious side effects in light of the severity of the disease and necessity of the treatment.

The terms “therapeutically effective” and “pharmacologically effective” are intended to qualify the amount of each agent which will achieve the goal of decreasing disease severity while avoiding adverse side effects such as those typically associated with alternative therapies. The therapeutically effective amount may be administered in one or more doses.

“Targeting,” as used herein, refers to the ability of the amine-passivated gold nanoparticles to be delivered to and preferentially accumulate in cancer tissue in a subject.

As used herein, “a detectably effective amount” of the imaging agent of the invention is defined as an amount sufficient to yield an acceptable image using equipment which is available for clinical use. A detectably effective amount of the imaging agent of the invention may be administered in more than one injection. The detectably effective amount of the imaging agent of the invention can vary according to factors such as the degree of susceptibility of the individual, the age, sex, and weight of the individual, idiosyncratic responses of the individual, and the dosimetry. Detectably effective amounts of the imaging agent of the invention can also vary according to instrument and film-related factors. Optimization of such factors is well within the level of skill in the art.

Amine-passivated Gold Nanoparticles

In one aspect, the invention provides an amine-passivated gold (Au) nanoparticle, comprising a gold nanoparticle passivated with a plurality of amine-containing drug or imaging agents. The amine-passivated gold nanoparticles of the present invention can provide several advantages. They can be synthesized in simple steps at low cost; off-the-shelf drugs can be loaded onto the nanoparticles without chemical modification; a significant amount of drug or imaging agent loading (e.g., about 100 compounds per nanoparticle); and are fairly uniform in size, ensuring consistent bio-availability and therapeutic or diagnostic effects. Amine-containing drugs or imaging agents are loaded on the gold nanoparticle via amine-Au interactions, creating a passivated nanoparticle, and are controllably displaced by thiols at the nano-core surface via ligand exchange upon reaching their target tissues. However, the amine-passivated gold nanoparticles are stable in plasma at physiological pH and in saline or cell culture under pH conditions ranging from a pH of 4 to a pH of 9.

The amine-passivated gold nanoparticles are water soluble, and formed of gold nanoparticle colloids which are passivated with the amine-containing drug or imaging agent, which cover and stabilize (i.e., passivate) the gold core. The term “passivated,” as used herein, refers to the protection and solubilization of the gold nanoparticle through formation of a layer over the nanoparticle. Amine-passivated refers to amine-containing drugs or imaging agents which passivate the gold nanoparticle by forming a layer in which the amine group associates with the nanoparticle. The amine-passivated gold nanoparticles are fairly uniform in size. For example, in some embodiments, the nanoparticles differ in size by a maximum of 1, 2, 3, 4, or 5 nanometers, in various embodiments. The amine-passivated gold nanoparticles are also relatively small in size. The amine-passivated gold nanoparticles have a size of about 20 nanometers or less. In some embodiments, the gold nanoparticles have a size of about 10 nanometers or less. In further embodiments, the gold nanoparticles have a size from about 1 to 5 nanometers, while in other embodiments the gold nanoparticles have a size of about 2 to 4 nanometers.

The amine-passivated gold nanoparticles are passivated with a plurality of amine-containing drugs or imaging agents. In some embodiments, the nanoparticles are passivated with from 10 to 200 amine-containing drugs or imaging agents, while in other embodiments the nanoparticles are passivated with from 50 to 150 amine-containing drugs or imaging agents. The amine-containing drugs or imaging agents can vary in size. Preferably, the amine-containing drugs or imaging agents have a size of 300 daltons or more, while in some embodiments the amine-containing drugs or imaging agents have a size from 300 to 1,000 daltons, while in further embodiments the amine-containing drugs or imaging agents have a size from 400 to 800 daltons. An amine-containing drug or imaging agent is an organic compound including at least one amine moiety. The amine can be a primary, secondary, or tertiary amine. However, in some embodiments, the amine-containing drugs include only drugs having a primary amine.

In some embodiments more than one type of amine compound is loaded on the amine-passivated gold nanoparticle. By including different types of compounds, different treatment and/or imaging strategies can be simultaneously implemented. For example, a gold nanoparticle can be loaded with different antitumor agents having a combined synergistic effect, or a gold nanoparticle including a cytotoxic agent can also include an imaging agent tracking by a visualization technique.

Amine-Containing Imaging Compounds

In some embodiments, the gold nanoparticle is passivated with an amine-containing imaging agent. The detectable group can be any material having a detectable physical or chemical property. Such detectable labels have been well-developed in the field of fluorescent imaging, magnetic resonance imaging, positive emission tomography, or immunoassays and, in general, most any label useful in such methods can be applied to the present invention, so long as it includes an amine group. Preferably, the amine-containing imaging agent also has a molecular weight of 300 daltons or more. Examples of imaging agents include fluorescent, MRI contrast agents, enzymatic moieties, or other suitable detectable labels. For example, in some embodiments, the imaging agent is an amine-containing dye molecule for fluorescent imaging. Examples of amine-containing dyes include aminocoumarin, folate-conjugated R-phycoerythrin, lucifer yellow, 4′,6-diamidino-2-phenylindole (DAPI), ethidium bromide, propidium iodide, and dihydrorhodamine 123. As indicated above, a wide variety of labels may be used, with the choice of label depending on sensitivity required, ease of conjugation with the compound, stability requirements, available instrumentation, and disposal provisions. Note also that in some embodiments, the gold nanoparticles themselves can be detected, and that many drugs will also fluoresce, particularly after release from the gold nanoparticles.

Means of detecting labels are well known to those of skill in the art. Thus, for example, where the label is a fluorescent label, it may be detected by exciting the fluorochrome with the appropriate wavelength of light and detecting the resulting fluorescence. The fluorescence may be detected visually, by means of photographic film, by the use of electronic detectors such as charge coupled devices (CCDs) or photomultipliers and the like. Similarly, enzymatic labels may be detected by providing the appropriate substrates for the enzyme and detecting the resulting reaction product.

In some embodiments, the method also includes the step of imaging the cancer tissue in the subject using an imaging device after administering a diagnostically effective amount of an amine-passivated gold nanoparticle to a subject. Examples of imaging methods include optical imaging, computed tomography, positive emission tomography, and magnetic resonance imaging.

Amine-Containing Drugs

In certain embodiments of the invention, the gold nanoparticles can be passivated by one or more amine-containing drugs. Amine-containing drugs are compounds that have a therapeutic effect when administered to a subject. Examples of amine-containing drugs include cytotoxic compounds, such as antibacterial, antiviral, or anticancer compounds that inhibit pathogen growth or promote pathogen death when proximate to or absorbed by an infected or cancerous cell. Suitable cytotoxic compounds include chemotoxic agents such as differentiation inducers, inhibitors and small chemotoxic drugs, toxin proteins and derivatives thereof. Preferably, the amine-containing drugs have a molecular weight of 300 daltons or more.

While it is not possible to list all of the amine-containing drugs that can be delivered using the amine-passivated gold nanoparticles of the invention, amine-containing drugs can be readily identified by review of the structure of the compound, and can be obtained from chemical texts such as the Merck Index. An amine-containing drug of interest can also be readily tested for its ability to passivate gold nanoparticles by preparing gold nanoparticles passivated with the drug of interest using the synthesis methods described herein.

Amine-containing anticancer drugs include anthracyclines such as daunorubicin, doxorubicin, idarubicin, epirubicin, and valrubicin; kinase inhibitors such as crizotinib, pazopanib, ibrutinib, and lenvatinib; nucleoside analogs such as cytarabine, decitabine, gemcitabine, cladribine, clofarabine, fludarabine, and vidarabine, as well as their and their mono-/pyro-/tri-phosphates; anti-metabolites such as methotrexate, permetrexed, aminopterin, and thioguanine; DNA alkylating agents such as dacarbazine, melphalan, and temozolomide; and other anticancer compounds such as lenalidomide.

Amine-containing antiviral drugs include nucleoside analogs such as anti-ebola agents such as BCX4430, anti-HIV agents such as emtricitabine, lamivudine, zalcitabine, and other antiviral agents such as abacavir, aciclovir, entecavir, as well as their mono-/pyro-/tri-phosphates.

Amine-containing antibacterial drugs include tetracyclines such as tetracycline, doxycycline, oxytetracycline, minocycline, demeclocycline, lymecycline, meclocycline, methacycline, roliteracycline, chlortetracycline, and tigcycline; aminoglycosides such as streptomycin, amikin, garamycin, kanamycin, neomycin, netilmicin, tobramycin, and paromomycin; ansamycins such as geldanamycin and herbimycin; carbacephem and loracarbef; carbapenem and doripenem; cephalosporins such as cefadroxil, cephalexin, cefaclor, cefoxitin, cefprozin, cefuroxime, cefixime, cefdinir, cefditoren, cefotaxime, cefpodoxime, ceftazidime, ceftibuten, ceftizoxime, ceftriaxone, cefepime, and ceftobiprole; glycopeptides such as teicoplanin, vancomycin, telavancin, and oritavancin; lipopeptide and daptomycin; monobactams such as aztreonam; penicillins such as ampicillin and amoxicillin; polypeptides such as polymyxin B, bacitracin, and colistin; fluoroquinolone such as gemifloxacin, trovafloxacin, and sparfloxacin; sulfonamides such as mafenide, sulfacetamide, sulfadiazine, sulfadimethoxine, sulfamethizole, sulfamethoxazole, sulfanilamide, sulfafurazole, and prontosil; anti-mycoplasms such as dapsone, capreomycin, ethionamide, isoniazid, and pyrazinamide; and other antibacterial agents such as arsphenamine trimethoprim.

Targeting Molecules

In some embodiments, a targeting molecule can be attached to the amine-passivated gold nanoparticle. By “targeting molecule,” what is meant herein is a compound that serves to target or direct the amine-passivated gold nanoparticles to a particular location, cell type, diseased tissue, or association. In general, the targeting molecule specifically binds a specific target epitope. “Specifically binds” means that non-target cells are either not specifically bound by the antibody or are only poorly recognized by the antibody. Thus, for example, antibodies, cell surface receptor ligands and hormones, lipids, sugars and dextrans, alcohols, peptides and nucleic acids may all be attached to localize or target the amine-passivated gold nanoparticle to a particular site. Preferably, when a targeting molecule is included, the gold nanoparticle includes from 1 to 10 targeting molecules. While too many targeting molecules will diminish the drug payload, in some embodiments up to 100 targeting molecules can be included per nanoparticle.

As used in this invention, the term “epitope” means any antigenic determinant on an antigen to which the antibody binds. Epitopic determinants usually consist of chemically active surface groupings of molecules such as amino acids or sugar side chains and usually have specific three dimensional structural characteristics, as well as specific charge characteristics. Epitopes of the invention can be present, for example, on cell surface receptors.

Epitopes to which tumor-specific antibodies bind are also well known in the art. For example, epitopes bound by the tumor-specific antibodies of the invention include, but are not limited to, those known in the art to be present on CA-125, gangliosides G(D2), G(M2) and G(D3), CD20, CD52, CD33, Ep-CAM, CEA, bombesin-like peptides, PSA, HER2/neu, epidermal growth factor receptor, erbB2, erbB3, erbB4, CD44v6, Ki-67, cancer-associated mucin, VEGF, VEGFRs (e.g., VEGFR3), estrogen receptors, Lewis-Y antigen, TGF 1, IGF-1 receptor, EGF, c-Kit receptor, transferrin receptor, IL-2R and CO17-1 Å.

The targeting molecule is linked to the surface of the amine-passivated gold nanoparticle through a thiol linkage. Most peptides include one or more cysteine amino acids that include a thiol group that will bond to the gold nanoparticle. Because the thiol groups have a higher affinity for the surface of the gold nanoparticle than amine groups, they will naturally displace a portion of the amine groups to form a more stable gold-thiol attachment in a spontaneous fashion. Thus, amine-passivated gold nanoparticles can be readily modified to include targeting molecules. Conjugation proceeds simply by mixing the targeting molecules to a solution including amine-passivated gold nanoparticles at desired targeting molecule-to-gold molar ratios, in contrast to the multi-step conventional linker chemistry. Targeting molecules lacking one or more thiol groups can be modified to include a thiol group. For example, peptide sequences can easily be modified to include a cysteine residue at the C-terminus.

In some embodiments, the targeting molecule is a peptide. For example, chemotactic peptides have been used to target tissue injury and inflammation, particularly by bacterial infection; see WO 97/14443, hereby expressly incorporated by reference in its entirety. Other examples of peptides that can be used as targeting molecules include granulocyte colony stimulating factor (GCSF), a GCSF mimetic peptide, erythropoietin, erythropoietin mimetic peptide, and a myeloid targeting cell penetrating peptide.

In some embodiments, the targeting molecule is all or a portion (e.g. a binding portion) of a ligand for a cell surface receptor. Suitable ligands include, but are not limited to, all or a functional portion of the ligands that bind to a cell surface receptor selected from the group consisting of insulin receptor (insulin), insulin-like growth factor receptor (including both IGF-1 and IGF-2), granulocyte colony stimulating factor receptor (GCSF), growth hormone receptor, glucose transporters (particularly GLUT 4 receptor), transferrin receptor (transferrin), epidermal growth factor receptor (EGF), low density lipoprotein receptor, high density lipoprotein receptor, leptin receptor, estrogen receptor (estrogen); interleukin receptors including IL-1, IL-2, IL-3, IL-4, IL-5, IL-6, IL-7, IL-8, IL-9, IL-11, IL-12, IL-13, IL-15, and IL-17 receptors, human growth hormone receptor, VEGF receptor (VEGF), PDGF receptor (PDGF), transforming growth factor receptor (including TGF- and TGF-), erythropoietin receptor (EPO and erythropoietin mimetic peptide), thrombopoietin receptor (TPO), ciliary neurotrophic factor receptor, prolactin receptor, and T-cell receptors. Receptor ligands include ligands that bind to receptors such as cell surface receptors, which include hormones, lipids, proteins, glycoproteins, signal transducers, growth factors, cytokines, peptide mimetics, and others.

In other embodiments, the targeting moiety is an antibody. The term “antibody” includes antibody fragments, as are known in the art, including Fab Fab₂, single chain antibodies (Fv for example), chimeric antibodies, etc., either produced by the modification of whole antibodies or those synthesized de novo using recombinant DNA technologies. In further embodiments, the antibody targeting moieties of the invention are humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin.

In some embodiments, the targeting molecule is intended for use in cancer treatment. A wide variety of tumor-specific antibodies are known to those skilled in the art. See Scott et al., Nature, 12, 278-287 (2012), the disclosure of which is incorporated herein by reference. For example, antibodies of the invention that bind to tumor cell epitopes include, but are not limited to, IMC-C225, EMD 72000, OvaRex Mab B43.13, 21B2 antibody, anti-human CEA, CC49, anti-ganglioside antibody G(D2) ch14.18, OC-125, F6-734, CO17-1A, ch-Fab-A7, BIWA 1, trastuzumab, rhuMAb VEGF, sc-321, AF349, BAF349, AF743, BAF743, MAB743, AB1875, Anti-Flt-4AB3127, FLT41-A, rituximab, tositumomab, Mib-1, 2C3, BR96, CAMPATH 1H, 2G7, 2A11, Alpha IR-3, ABX-EGF, MDX-447, SR1, Yb5.b8, 17F.11, anti-p75, anti-p64 IL-2R and MLS 102.

A wide variety of tumor-specific antibodies are known in the art, such as those described in U.S. Pat. Nos. 6,197,524, 6,191,255, 6,183,971, 6,162,606, 6,160,099, 6,143,873, 6,140,470, 6,139,869, 6,113,897, 6,106,833, 6,042,829, 6,042,828, 6,024,955, 6,020,153, 6,015,680, 5,990,297, 5,990,287, 5,972,628, 5,972,628, 5,959,084, 5,951,985, 5,939,532, 5,939,532, 5,939,277, 5,885,830, 5,874,255, 5,843,708, 5,837,845, 5,830,470, 5,792,616, 5,767,246, 5,747,048, 5,705,341, 5,690,935, 5,688,657, 5,688,505, 5,665,854, 5,656,444, 5,650,300, 5,643,740, 5,635,600, 5,589,573, 5,576,182, 5,552,526, 5,532,159, 5,525,337, 5,521,528, 5,519,120, 5,495,002, 5,474,755, 5,459,043, 5,427,917, 5,348,880, 5,344,919, 5,338,832, 5,298,393, 5,331,093, 5,244,801, and 5,169,774. See also The Monoclonal Antibody Index Volume 1: Cancer (3rd edition). Accordingly, tumor-specific antibodies of the invention can recognize tumors derived from a wide variety of tissue types, including, but not limited to, breast, prostate, colon, lung, pharynx, thyroid, lymphoid, lymphatic, larynx, esophagus, oral mucosa, bladder, stomach, intestine, liver, pancreas, ovary, uterus, cervix, testes, dermis, bone, blood and brain.

Preparation of Amine-Passivated Gold Nanoparticles

Another aspect of the invention provides a method of making an amine-passivated gold nanoparticle, comprising combining an amine-containing drug or imaging agent with HAuCl₄ and a reducing agent in a polar organic solvent at a temperature from −80 to 20° C. for a time sufficient to form amine-passivated gold nanoparticles. Examples of polar organic solvents include DMSO, methanol, acetonitrile, ethanol, and tetrahydrofuran. Examples of reducing agents include various borohydrides such as sodium borohydride, tetramethylammonium borohydride, tetraethylammonium borohydride, and sodium triacetoxyborohydride. It is generally preferable to agitate the reaction mixture by, for example, stirring the reaction. The reaction time, temperature, and solvent can be varied depending on the particular amine-containing drug or imaging agent, and the desired nature of the amine-passivated gold nanoparticles. For example, in some embodiments, the time sufficient to form amine-passivated gold nanoparticles is about one hour. The amine-passivate gold nanoparticles prepared can include any of the sizes and amine-containing drug or imaging agents described herein.

When it is desired that the amine-passivating gold nanoparticle also includes a targeting molecule, the reaction further includes the step of reacting the amine-passivated gold nanoparticle with a thiol-containing targeting molecule. Conjugation proceeds by mixing the target molecules into the solution including the amino-passivated gold nanoparticles at the desired molar ratios. The targeting molecule-to-gold molar ratio, as well as the conjugation conditions including solvent, temperature, and pH, can be readily determined by one skilled in the art without undue experimentation.

For example, amine-passivated gold nanoparticles can be prepared as follows. Amine-containing drug molecules and Au precursor (HAuCl₄) are mixed and reacted in one flask. Important parameters in the inventors one-pot synthesis have been identified, and include themolecular weight of the amine-containing molecule as ligands; reaction solvent with different polarity, reducing agent (in the order of high to low in reducing strength: sodium borohydride, tetramethylammonium borohydride, tetraethylammonium borohydride, sodium triacetoxyborohydride), and temperature (from room temperature to −80° C.). By adjusting these conditions, amine-passivated gold nanoparticles including a wide variety of amine-containing drugs were obtained by the inventors, including gold nanoparticles loaded with anticancer drugs.

Detailed structural information of the prepared amine-passivated gold nanoparticles can be assessed using a range of physical chemistry techniques including optical spectroscopy, TEM (transmission electron microscopy), mass spectrometry—specifically MALDI (matrix assisted laser disorption ionization) and ESI (electrospray ionization), elemental analysis (CHN, carbon-hydrogen-nitrogen, analysis combined with ICP-MS, inductively-coupled plasma mass spectrometry), 1H-NMR (proton nuclear magnetic resonance) spectroscopy, and FTIR (Fourier transform infra-red) spectroscopy.

Cancer Treatment Using Amine-Passivated Gold Nanoparticles

An additional aspect of the present invention provides a method of treating cancer in a subject identified as having cancer by administering to the subject a therapeutically effective amount of an amine-passivated gold nanoparticle, and in particular a gold nanoparticle passivated with an amine-containing anticancer agent. A variety of amine-containing anticancer agents are described herein.

In some embodiments, the amine-passivated gold nanoparticle is used to target tissue in a subject without the use of a targeting moiety based on the ability of the nanoparticles to preferentially accumulate in certain tissues. In particular, the gold nanoparticles have been shown to preferentially accumulate in diseased tissue, such as cancer tissue or inflamed tissue (e.g., atherosclerotic blood vessels) that is more permeable than regular tissue. The present invention also takes advantage of differences between cancer and normal cells for controlled delivery of drugs to cancer cells. One such difference is the ubiquitously elevated glutathione expression in cancer cells. The high level of glutathione expression by cancer cells encourages ligand exchange with the amine-passivated gold nanoparticles, releasing the amine-containing drugs or imaging agents. See Egusa et al., Exp Bio Med 239, 853-61 (2014), the disclosure of which is incorporated herein by reference.

Gold nanoparticles including anticancer compounds can be used to treat a variety of different types of cancer. “Cancer” or “malignancy” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize) as well as any of a number of characteristic structural and/or molecular features. A “cancer cell” refers to a cell undergoing early, intermediate or advanced stages of multi-step neoplastic progression. The features of early, intermediate and advanced stages of neoplastic progression have been described using microscopy. Cancer cells at each of the three stages of neoplastic progression generally have abnormal karyotypes, including translocations, inversion, deletions, isochromosomes, monosomies, and extra chromosomes. Cancer cells include “hyperplastic cells,” that is, cells in the early stages of malignant progression, “dysplastic cells,” that is, cells in the intermediate stages of neoplastic progression, and “neoplastic cells,” that is, cells in the advanced stages of neoplastic progression. Examples of cancers are sarcoma, breast, lung, brain, bone, liver, kidney, colon, and prostate cancer. In some embodiments, the amine-passivated gold nanoparticles including anticancer agents are used to treat cancer tissue selected from the group consisting of colon cancer, brain cancer, breast cancer, fibrosarcoma, and squamous carcinoma. A preferred type of cancer suitable for treatment using the amine-passivated gold nanoparticles is acute myeloid leukemia (AML). For example, direct intracellular delivery of phosphorylated cytarabines by the gold nanoparticles offers an important opportunity in deoxycytidine kinase (dCK)-mutated AML treatment, bypassing dCK metabolism.

The method includes treating a subject that has been identified as having cancer. A subject can be identified as having cancer using a wide variety of diagnostic criteria known to those skilled in the art. Most cancers are initially recognized either because of the appearance of signs or symptoms or through screening. A definitive diagnosis requires the examination of a tissue sample, typically obtained by a biopsy, by a pathologist. Subjects with suspected cancer are investigated with medical tests. These commonly include blood tests, X-rays, CT scans and endoscopy.

In some embodiments, the cancer treated is Acute myeloid leukemia. Acute myeloid leukemia treatment remains a major challenge in oncology, with an estimated 18,860 deaths and 10,460 new cases in 2014 in the United States alone. Cytotoxic chemotherapy is the mainstay in AML treatment today, wherein escalated dose is often necessary to induce remission. However this is done at the cost of enormous burden on the patients: treatment causes fatal exacerbations of low blood counts in up to 29% of AML patients. The amine-passivated gold nanoparticles described herein could be substantially address this problem by targeting drugs selectively to malignant myeloid cells, thereby minimizing exposure of normal stem/progenitor cells to the drugs. For example, the amine-passivated gold nanoparticles could include targeting molecules specific for CD33, which is overexpresed in AML.

The amine-passivated gold nanoparticles can include any of the amine-containing drugs or imaging agents described herein, and can have any of the sizes described herein, such as having a size from 1 to 5 nanometers. In addition, in some embodiments, the amine-passivated gold nanoparticle also includes a targeting molecule bonded to the nanoparticle through a thiol linkage. The targeting molecule can be selected to direct the amine-passivated gold nanoparticles to the cancer present in the subject, which has previously been biopsied and evaluated using methods known to those skilled in the art. For example, in some embodiments, the targeting molecule is selected from the group consisting of granulocyte colony stimulating factor (GCSF), a GCSF mimetic peptide, erythropoietin, and a myeloid targeting cell penetrating peptide.

Administration and Formulation of Amine-Passivated Gold Nanoparticles

In some embodiments, the amine-passivated gold nanoparticle is administered together with a pharmaceutically acceptable carrier to provide a pharmaceutical formulation. Pharmaceutically acceptable carriers enable the amine-passivated gold nanoparticle to be delivered to the subject in an effective manner while minimizing side effects, and can include a variety of diluents or excipients known to those of ordinary skill in the art. Formulations include, but are not limited to, those suitable for oral, rectal, vaginal, topical, nasal, ophthalmic, or parental (including subcutaneous, intramuscular, intraperitoneal, intratumoral, and intravenous) administration. For example, for parenteral administration, isotonic saline is preferred. For topical administration, a cream, including a carrier such as dimethylsulfoxide (DMSO), or other agents typically found in topical creams that do not block or inhibit activity of the compound, can be used. Other suitable carriers include, but are not limited to, alcohol, phosphate buffered saline, and other balanced salt solutions.

The formulations may be conveniently presented in unit dosage form and may be prepared by any of the methods well known in the art of pharmacy. Preferably, such methods include the step of bringing the amine-passivated gold nanoparticle into association with a pharmaceutically acceptable carrier that constitutes one or more accessory ingredients. In general, the formulations are prepared by uniformly and intimately bringing the active agent into association with a liquid carrier, a finely divided solid carrier, or both, and then, if necessary, shaping the product into the desired formulations. The methods of the invention include administering to a subject, preferably a mammal, and more preferably a human, the composition of the invention in an amount effective to produce the desired effect. The formulated amine-passivated gold nanoparticles can be administered as a single dose or in multiple doses.

Useful dosages of the active agents can be determined by comparing their in vitro activity and the in vivo activity in animal models. Methods for extrapolation of effective dosages in mice, and other animals, to humans are known in the art; for example, see U.S. Pat. No. 4,938,949. An amount adequate to accomplish therapeutic or prophylactic treatment is defined as a therapeutically- or prophylactically-effective dose. In both prophylactic and therapeutic regimes, agents are usually administered in several dosages until an effect has been achieved. Effective doses of the amine-passivated gold nanoparticle vary depending upon many different factors, including means of administration, target site, physiological state of the patient, whether the patient is human or an animal, other medications administered, and whether treatment is prophylactic or therapeutic.

For administration for targeting or imaging in a subject utilizing an amine-passivated gold nanoparticle, the dosage of the drug or imaging agent ranges from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. A suitable amount of nanoparticle is used to provide the desired dosage. An exemplary treatment regime entails administration once per every two weeks or once a month or once every 3 to 6 months. The amine-passivated gold nanoparticle is usually administered on multiple occasions. Alternatively, the amine-passivated gold nanoparticle can be administered as a sustained release formulation, in which case less frequent administration is required. In therapeutic applications, a relatively high dosage at relatively short intervals is sometimes required until progression of the disease is reduced or terminated, and preferably until the patient shows partial or complete amelioration of symptoms of disease. Thereafter, the patent can be administered a prophylactic regime.

The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

For parenteral administration, compositions of the invention can be administered as injectable dosages of a solution or suspension of the substance in a physiologically acceptable diluent with a pharmaceutical carrier that can be a sterile liquid such as water oils, saline, glycerol, or ethanol. Additionally, auxiliary substances, such as wetting or emulsifying agents, surfactants, pH buffering substances and the like can be present in compositions. Other components of pharmaceutical compositions are those of petroleum, animal, vegetable, or synthetic origin, for example, peanut oil, soybean oil, and mineral oil. In general, glycols such as propylene glycol or polyethylene glycol are preferred liquid carriers, particularly for injectable solutions.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Ligand Exchange on Gold Nanoparticles for Drug Delivery and

Enhanced Therapeutic Index Evaluated in Acute Myeloid Leukemia Models

Glutathione-mediated ligand exchange (Harmsen et al., Bioconjugate Chem; 22(4):540-5 (2011)) could be a promising scheme of controlled intracellular payload release in vivo, exploiting higher level of cell-associated glutathione compared to that of plasma. Anderson M E., Chem Biol Interact; 111-112:1-14 (1998) Because ligand exchange is based upon competitive affinity to Au surface between the original and the incoming ligands, a wider range of payload release kinetics would become available by exploiting Au nanoparticles directly passivated with payloads as ligands, through functional groups with weaker affinity to Au than that of thiols such as amines, carboxyls, and phosphines etc. (principle of ligand exchange, utilizing the differential in the affinity to Au, has been described: Woehrle et al., J Phys Chem B; 106:9979-81 (2002); Brown L O, Hutchison J E., J Am Chem Soc; 119:12384-5 (1997)). Passivation relying on weak Au affinity would seem at first to inhibit the formation of Au nanoparticles, or to sacrifice the colloidal stability especially under physiologically relevant conditions. Here, the versatile synthesis of small (˜2.5 nm) water-soluble Au nanoparticles that are directly passivated with amine-containing molecules is described. Taking methotrexate-passivated Au nanoparticles (Au:MTX) as an example, the stability in physiological environments was examined, and the results demonstrate the payload (i.e. MTX) release triggered by glutathione. Furthermore, the inventors have demonstrated improved therapeutic index in vitro using a cancer/normal cell comparison, and in vivo using a murine xenotransplant model of primary human acute myeloid leukemia (AML), a cancer which is disseminated without large tumor masses, and therefore in which there should be less benefit, if any, from EPR effect.

Materials and Methods

Materials

Au:MTX Synthesis and Characterization

In a typical synthesis of water soluble nanoparticles Au:MTX, 5 μmol of HAuCl₄ and 25 μmol of MTX were dispersed with brief sonication in 2 mL methanol in a tri-neck 15 mL flask on ice water (0° C.) bath under Ar purge. After 1 hour of stirring at 700 rpm, 0.5 mL of freshly prepared 0.11 M NaBH₄ on ice was added drop-wise at 1200 rpm. After ˜1 hour, stirring speed was reduced to 700 rpm and kept for additional ˜30 minutes. Scale-up of the reaction has been confirmed up to 20-fold, yielding consistent results by transmission electron microscopy (TEM) characterization.

Au:MTX Nanoparticle Purification and Determination of Drug Loading

The reacted solution was centrifuged at 4° C. at 16,100×g for 15 minutes twice, with the addition of 4:1 (volume) mixture of methanol and water, and ethanol respectively. This set of two step centrifugation was repeated at least 3 times, and then the precipitate was re-dispersed in water to obtain aqueous solutions. The aqueous solution was further purified using 3 k ultra centrifugal filter (Millipore, Billerica, Mass.). Following the sets of centrifugations of Au:MTX as described above, purity of Au:MTX was verified by sodium dodecyl sulfate polyacrylamide gel electrophoresis (SDS-PAGE). Elemental analytic measurements (Galbraith Laboratories, Inc., Knoxville, Tenn.) including carbon-hydrogen-nitrogen analysis (CHN) and inductively coupled plasma mass spectrometry (ICP-MS) confirmed the purity of the compound, where absence of organics other than MTX was verified via CHN, and Au-to-MTX ratio [Au]:[C]:[N] was determined via CHN and ICP-MS.

Ligand Exchange Induced by Glutathione and Release of MTX from Au:MTX

Aqueous solution of Au:MTX (1 mM, in Au molar amount) was mixed with glutathione (GSH; 0.5, 1, or 5 mM) under 700 rpm stirring at room temperature, to induce MTX-to-GSH ligand exchange. Aliquot was diluted in water and extinction spectrum was measured using ultraviolet-visible (UV-Vis) optical spectroscopy. The amount of released free MTX in solution was evaluated by extinction spectra at 90 minutes of stirring. The ligand exchange process was monitored up to 15 hours.

Evaluation of Au:MTX Nanoparticle Efficacy In Vitro

In order to evaluate the efficacy of Au:MTX in vitro, a human AML cell line THP-1 and human normal hematopoietic stem/progenitor cells (HSPCs) were used. HSPCs were isolated from umbilical cord blood via a CD34+ magnetic cell sorting (CD34 MicroBead Kit #130-046-702, Miltenyl Biotec, Auburn, Calif.). THP-1 cells were cultured in Roswell Park Memorial Institute 1640 (RPMI, Life Technologies, Carlsbad, Calif.) with 10% fetal bovine serum (FBS, Life Technologies), and HSPCs were cultured in Iscove's Modified Dulbecco's Medium (IMDM, Life Technologies) with 10% FBS and cytokines (10 ng/mL of SCF, TPO, and FLT3; 5 ng/mL of IL-3 and IL-6, PeproTech, Rockey Hill, N.J.), respectively, using 24-well cell culture plates with 0.7 mL of culture media per well (at 37° C. with 5% CO₂ and >95% humidity). Cell viability was confirmed to be >95% using cell viability analyzer (Vi-Cell, Beckman Coulter, Brea, Calif.) immediately before the experiments. Then the cells were treated with phosphate buffer saline (PBS, Life Technologies), MTX, aqueous solutions of equimolar Au:MTX, and/or equimolar Au:FOL (folic acid-passivated Au nanoparticles). Cell cultures were split two-fold after 72 hours by replenishing each well with fresh media. Cell density and viability were measured every 24 hours using the cell viability analyzer. For the evaluation of cellular uptake of Au:MTX, THP-1 cells were treated with 500 nM Au:MTX for 4 hours via incubation at the culturing conditions, then the cells were centrifuged and the resulting pellets were washed with PBS and dispersed in a standard TEM fixative (100 mM sodium cacodylate buffer with 4% paraformaldehyde and 2.5% glutaraldehyde), resin sectioned and mounted on nickel TEM grid. Thus prepared specimens were silver-enhanced (following the protocol associated with Silver Enhancer Kit #SE100, Sigma-Aldrich), and imaged by TEM.

Au:MTX Treatment on Murine Models and Evaluation of Tumor Burden

The animal experiments were conducted with the approval of the Cleveland Clinic Foundation's Institutional Animal Care and Use Committees (IACUC). Primary AML cells were transplanted in 6 week old non-obese diabetic severe combined immunodeficiency gamma (NSG) mice by tail vein intravenous (i.v.) injection. Five days post-transplantation, treatments with Au:MTX, along with MTX (as drug-only control), Au:FOL (as nanoparticle-only control), and PBS were initiated (5 mice per group, total of 20 mice). Mice were treated by tail vein i.v. injection metronomically twice per week under daily surveillance. Aqueous solutions of Au:MTX and Au:FOL, as well as stock solution of MTX in dimethyl sulfoxide (0.1 μL per gram of mouse weight per treatment), were diluted with PBS for i.v. injections. Mice were sacrificed according to the IACUC-approved protocol, when the PBS-treated group showed clear signs of anemia and distress. Murine bone marrows were analyzed by flow cytometry (CYTOMICS FC 500, Beckman Coulter), where the populations of human AML cells and mouse hematopoietic cells were determined by staining with ECD-conjugated anti-human CD45 monoclonal antibody (mAb) and APC-conjugated anti-mouse CD45 mAb, respectively (Beckman Coulter). Spleens, livers, and intestines were harvested and fixed in 4% paraformaldehyde and embedded in a paraffin block. The tissues were sectioned (5 μm thick), hydrated via immersions in xylene (3 times) and ethanol (100%, 95%, and 75% successively), then silver-enhanced and co-stained with hematoxylin and/or eosin.

Glutathione Assay of Cancer and Normal Hematopoietic Lineage Cells

Viability of THP-1, primary AML cells, and normal HSPCs was confirmed to be >95% using a cell viability analyzer immediately before experiments. Intracellular glutathione concentration was measured using ˜10⁷ cultured cells (following the protocol associated with the Glutathione Assay Kit #CS0260, Sigma-Aldrich).

Results

Synthesis of Au Nanoparticles Passivated by Amine-Containing Molecules

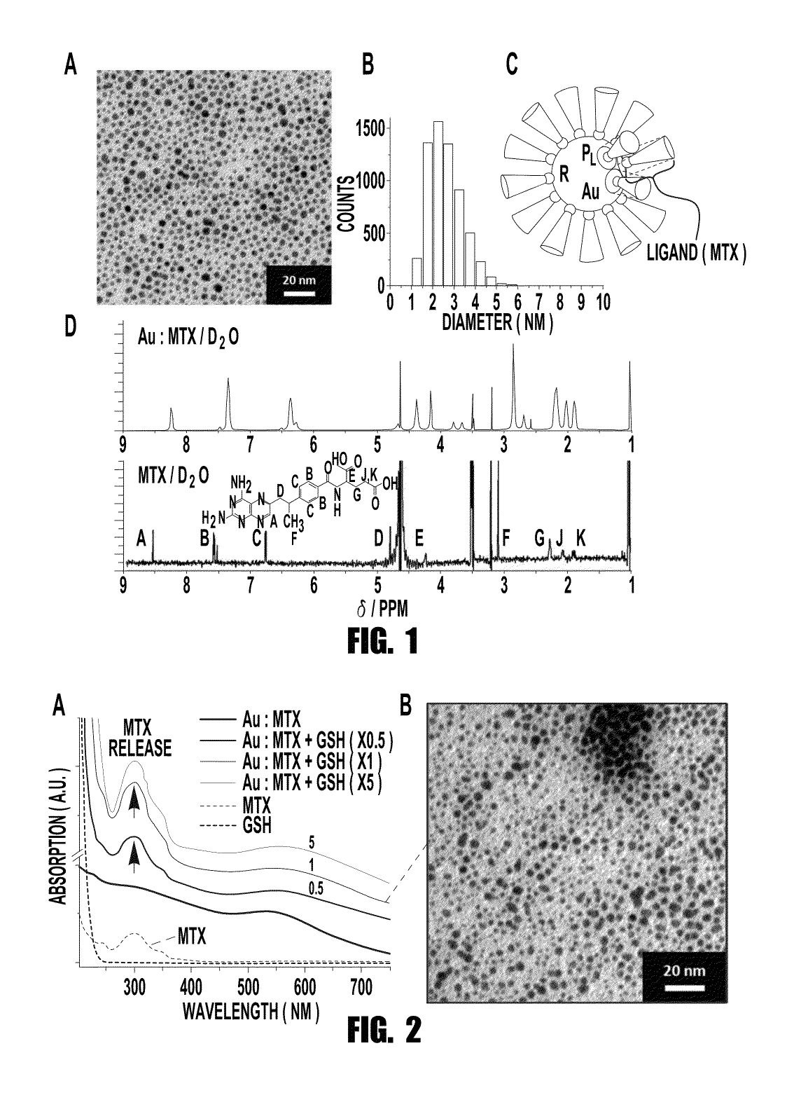

Compared to the extensive library of thiol-passivated nano-materials, there are fewer examples of amine-passivated nanoparticles, particularly water-soluble amine-Au nanoparticles. Rai et al., J Mater Chem; 20:6789-98 (2010); Porta et al., Langmuir; 24:7061-4 (2008). Using a variation of the method described by Schaaf et. al. for synthesizing thiol-passivated ultra-small Au nanoparticles (Chen et al., Science; 280:2098-101 (1998); Schaaff et al., J Phys Chem B; 102:10643-6 (1998)) and of the original method of Brust et. al. (Brust et al., J Chem Soc, Chem Commun: 801-2 (1994)), the inventors were able to synthesize water-soluble Au nanoparticles directly passivated with a monolayer of the chemotherapeutic methotrexate (Au:MTX) (FIG. 1(a-c); 2.6±0.7 nm in diameter, measured by transmission electron microscopy, or TEM), without necessitating post-synthesis chemistry. Proton nuclear magnetic resonance (¹H-NMR; Bruker, 600 MHz) measurements of Au:MTX in D₂O indicated large change of chemical shift (in FIG. 1(d), a: Δδ0.30 ppm; b: Δδ0.23 ppm; c: Δδ0.39 ppm) in the aromatic ring region of MTX. An aqueous solution of Au:MTX was stable when stored at 4° C., and no significant change was observed over 10 months with TEM and optical spectroscopy (the structural stability of amine-Au nanoparticles in organic phase has been reported by Heath et. al.). Heath et al., J Phys Chem B; 101:189-97 (1997); Leff et al., Langmuir; 12:4723-30 (1996).

In addition, to demonstrate the wide applicability of the synthesis protocol, the inventors conducted a systematic study on the formation of stable Au nanoparticles using amine-containing molecules with molecular weights (m.w.) ranging from 146.19 (lysine) to 785.55 (flavin adenosine dinucleotide, or FAD), using the same conditions. The results are summarized in Table 1. Despite the relatively weak affinity to Au, stable nanoparticles were formed with numerous amine-containing ligands, many of which are drugs in common use. The details of ligand-to-Au interactions require further elucidation. Of particular note, the nanoparticle formation was clearly dependent on the m.w. of the amine ligands: Ligands with m.w. <300 tended to not form colloidally dispersed nanoparticles. The existence of m.w. threshold can be attributed to the effective Au nanoparticle surface coverage for stable colloidal suspension (e.g. reviewed by Templeton et. al., Acc Chem Res; 33:27-36 (2000)), and the value of the threshold contrasts with the synthesis of thiol-passivated Au nanoparticles, where the threshold is much known to be smaller at m.w. <100, presumably due to strong thiol-Au affinity.

TABLE 1
Formation of monolayer-protected Au nanoparticles.
Amine-containing compounds are shown in the order
according to molecular weight.

| | amine compound | m.w. | nanoparticle |
|---|---|---|---|
| 1 | lysine | 146.19 | did not form |
| 2 | methionine | 149.21 | did not form |
| 3 | dopamine | 153.18 | did not form |
| 4 | arginine | 174.2 | did not form |
| 5 | deoxycytidine | 227.27 | did not form |
| 6 | cytidine | 243.22 | did not form |
| 7 | cytarabine (Ara-C) | 243.22 | did not form |
| 8 | deoxyadenosine | 251.24 | did not form |
| 9 | adenosine | 267.24 | did not form |
| 1 | deoxyguanosine | 267.24 | did not form |
| 1 | guanosine | 283.24 | 2.7 ± 1.6 nm |
| 1 | thiamine | 300.81 | did not form |
| 1 | cytidine monophosphate | 323.2 | 4.5 ± 2.8 nm |
| 1 | adenosine monophosphate | 347.22 | did not form |
| 1 | guanosine monophosphate | 363.22 | 2.6 ± 1.0 nm |
| 1 | cytidine diphosphate | 403.18 | 2.5 ± 0.8 nm |
| 1 | thyamine pyrophosphate | 425.31 | 3.1 ± 1.3 nm |
| 1 | adenosine diphosphate | 427.2 | 2.3 ± 0.7 nm |
| 1 | folic acid | 441.4 | 2.6 ± 1.1 nm |
| 2 | guanosine diphosphate | 443.2 | 2.4 ± 1.0 nm |
| 2 | tetracycline | 444.44 | 1.6 ± 0.7 nm |
| 2 | doxycycline | 444.44 | 1.7 ± 0.7 nm |
| 2 | methotrexate (MTX) | 454.44 | 2.6 ± 0.7 nm |
| 2 | methotrexate dimethyl ester | 482.49 | 2.2 ± 0.6 nm |
| 2 | cytidine triphosphate | 483.16 | 2.6 ± 0.9 nm |
| 2 | adenosine triphosphate | 507.18 | 2.1 ± 0.7 nm |
| 2 | guanosine triphosphate | 523.18 | 2.3 ± 0.7 nm |
| 2 | doxorubicin | 543.52 | 3.3 ± 1.2 nm |
| 2 | streptomycin | 581.57 | 2.5 ± 1.2 nm |
| 3 | flavin adenine dinucleotide (FAD) | 785.55 | 4.2 ± 1.6 nm |

Ligand Exchange on Au:MTX and Payload Release

An important property of Au:MTX is the possibility of controlled MTX release triggered by ligand exchange with thiols. To illustrate this, Au:MTX was mixed and stirred (700 rpm, room temperature) with varied concentrations of glutathione (GSH, Au-to-GSH molar ratio at 0.5, 1, and 5) in water to induce ligand exchange. GSH was chosen as the exchanging thiol, because of its ubiquitous endogenous abundance within bio-organisms and its potential relevance to MTX release in vivo as well as in vitro. MTX-to-GSH ligand exchange was verified: the free MTX in solution at 90 minutes of stirring was measured via ultraviolet-visible (UV-Vis) spectroscopy, and the release of MTX increased according to the GSH concentration (FIG. 2(a)). Au-to-GSH ratio c.a. >1 saturated the exchange. Also, no significant increase in released MTX was observed after 90 minutes. Au nanoparticle core size was largely preserved in this ligand exchange (FIG. 2(b)), although some degree of aggregation was observed.

Stability of Au:MTX in Physiological Conditions

Stability of Au:MTX was examined under physiologically relevant conditions (FIG. 3). Au:MTX was surprisingly stable at pH 4-9, and no precipitation or change in the color of solution was observed over 48 hours (FIG. 3(a)). At pH 2-3, Au:MTX precipitated within 2 hours at room temperature. The supernatant of Au:MTX (98 μM in Au molar amounts) precipitated at pH 3 contained ˜10 μM MTX that was released as a result of nanoparticle aggregation (FIG. 3(b)). It is noted that the gastric environment is at pH ˜1.5-3.5, and that the acidity in cell lysosome is at pH ˜5. Au:MTX was stable in saline solutions or in the presence of proteins (FIG. 3(c)). No precipitation was observed in phosphate buffer saline (PBS), in an albumin solution (bovine serum albumin, or BSA, 35 g/L), or in serum (fetal bovine serum, or FBS, 100%).

Enhanced Efficacy of Au:MTX In Vitro

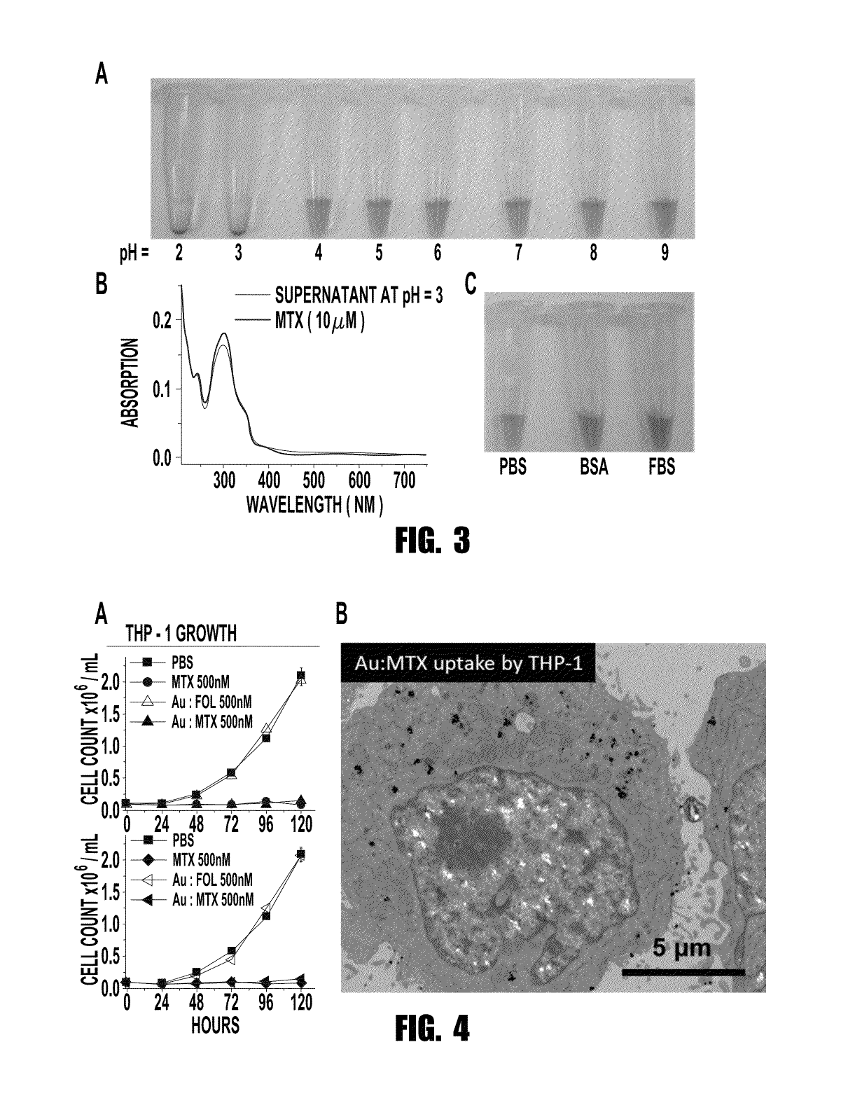

The inventors characterized Au:MTX as drug delivery vehicles and evaluated in vitro therapeutic effects. Au:MTX was purified via sets of centrifugal precipitations, and the Au-to-MTX molar ratio (drug loading onto Au nanoparticle) was measured by elemental analyses (carbon-hydrogen-nitrogen analysis, or CHN; and inductively coupled plasma mass spectrometry, or ICP-MS). CHN analysis yielded C: N=1:0.405, in good agreement with MTX chemical formula. Au-to-MTX molar ratio was determined as [Au]:[MTX]=9.8:1, i.e. one nanoparticle on average carried ˜75 MTX molecules (assuming ˜1.44 Å as atomic radius of Au and uniform surface coverage, as illustrated in FIG. 1(c)). This ratio was consistent with the spectroscopic observation in FIG. 3(b), when MTX was fully released due to nanoparticle aggregation and precipitation. This ratio was used in the following experiments to determine the MTX drug payload in Au:MTX, such that the net amount of MTX was the same as that of MTX-alone treatment. Drug delivery by Au:MTX was evaluated using a human AML cell line THP-1. THP-1 cells were treated with 50 and 500 nM concentrations (in molar amount of MTX, as determined by the elemental analysis) of Au:MTX, in comparison to treatments with buffer (PBS, as vehicle control), equivalent molar amounts of MTX alone, and folic acid-passivated Au nanoparticles (Au:FOL). Au:FOL, with the diameter 2.6±1.1 nm measured by TEM, is structurally similar to Au:MTX and was therefore used as an “Au nanoparticle without active drug” control. THP-1 growth curves indicate similar efficacy of MTX and Au:MTX treatments at 50 and 500 nM concentrations, thus confirming effective delivery of MTX by nanoparticles (FIG. 4(a)). It is important to note that Au:FOL did not exhibit significant effect on cell growth, suggesting very low toxicity caused by Au nanoparticle cores alone. Cellular uptake of Au:MTX was examined in vitro (FIG. 4(b)). TEM images were taken after THP-1 cells were incubated for 4 hours with 500 nM-MTX equimolar concentration of Au:MTX, and a majority of Au nanoparticles were found dispersed within the cells without forming large-scale aggregations.

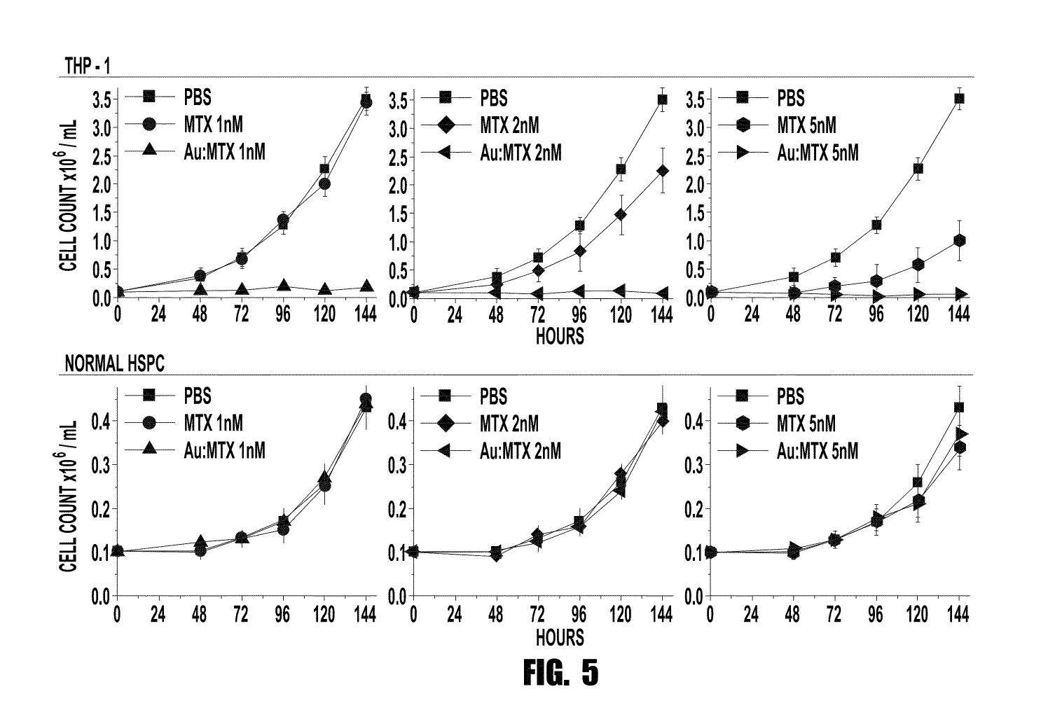

Enhanced efficacy of Au:MTX was validated in vitro using a human AML cell line THP-1 and normal hematopoietic stem/progenitor cells (HSPCs), as an appropriate normal counterpart to the myeloid cancer cells. THP-1 and HSPCs were treated with 1, 2, and 5 nM concentrations of Au:MTX and MTX (FIG. 5). Au:MTX exhibited elevated potency, and therefore enhanced drug delivery, compared to MTX-alone. Au:MTX completely inhibited THP-1 cell growth at 1-5 nM, whereas MTX-alone treatments resulted in dose-dependent suppression. On the other hand, HSPC growth was not significantly affected either by Au:MTX and MTX treatments within the same 1-5 nM dose range. Thus, in comparison to MTX-alone, Au:MTX can produce more selective growth inhibition of cancer (THP-1) cells while sparing normal HSPCs in vitro.

Efficacy and Safety of Au:MTX Nanoparticles In Vivo

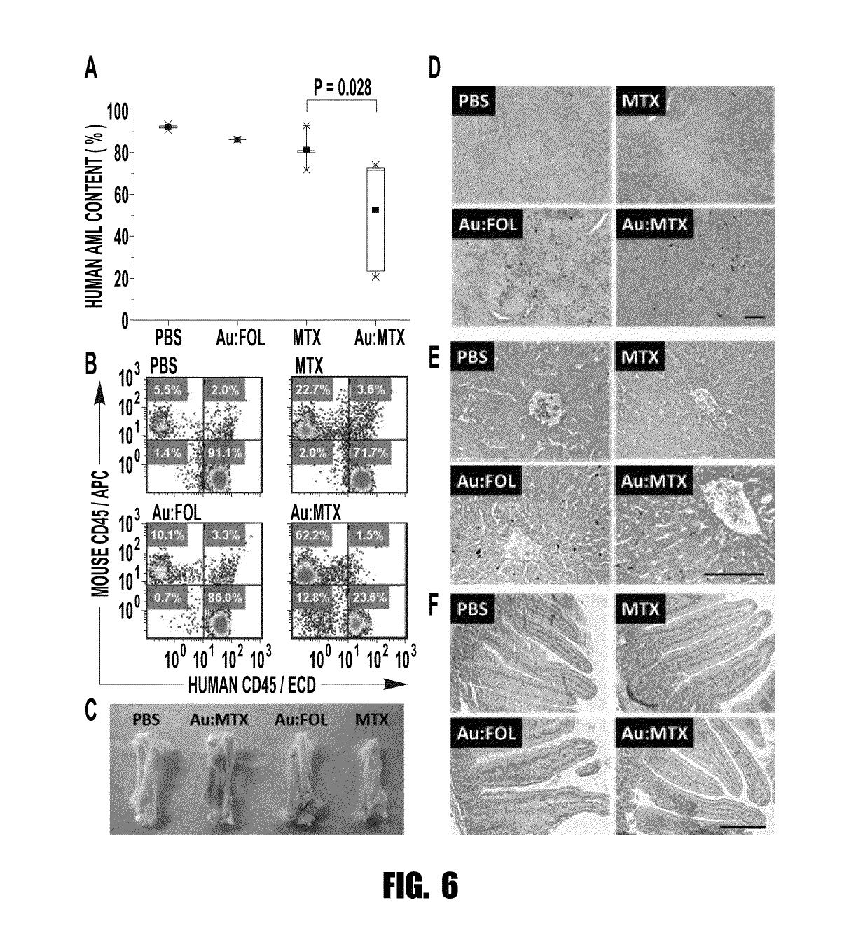

In order to evaluate the therapeutic index of Au:MTX in vivo, efficacy as well as safety were assessed in a murine xenotransplant model of primary human AML (using AML cells obtained from a patient). This model is a relatively faithful representation of the actual human disease, with diffuse infiltration of the bone marrow by human leukemia cells, splenomegaly, and fatality from cytopenia. Au:MTX treatment was compared to PBS, MTX alone, and Au:FOL treatments, all administered intravenously (i.v.) 2×/week by tail vein injection. Based on the literature (van de Steeg et al., Drug Metab Dispos; 37:277-81 (2009)), the inventors chose the dose of 0.25 mg/kg MTX, and accordingly the dose of Au:MTX and Au:FOL calculated from the drug loading (drug-to-Au molar ratio determined by the elemental analyses). Mice were monitored daily and euthanized at week 6 when the PBS control group demonstrated distress from anemia. Amongst these treatment groups, Au:MTX-treated mice exhibited the least sign of anemia, the consistently lower bone marrow leukemia cell burden compared to other treatment groups as quantified by flow-cytometry (FIG. 6(a-c), and the lowest splenic leukemia cell burden quantified by weight and histological examination (FIG. 6(d)). There was no evidence of increased toxicity from Au:MTX compared to the other treatments based on the weight, appearance, and behavior of the mice, or in histological examination of the liver and the rapidly proliferating cells, e.g., intestinal endothelium (FIG. 6(e, f)). Thus, Au:MTX treatment can induce a superior therapeutic index compared to MTX alone or the other treatment arms.

Discussion

Enhanced therapeutic index of Au:MTX in our primary AML model is a result of the effective delivery of MTX to AML cells compared to normal cells. The detailed mechanistic elucidation of the observed cancer selectivity necessitates further investigation. One contribution could originate from the difference in the rate of uptake among cell types, or the difference between folate receptor-mediated MTX transport and endocytotic uptake of Au:MTX. Thomas et al., Mol Pharm; 9(9):2669-76 (2012); Jackman et al., Adv Drug Deliv Rev; 56:1111-25 (2004). Another contribution could be the consequence of glutathione-mediated in-situ ligand exchange of Au:MTX, and the elevated level of glutathione in AML cells resulting in facilitated MTX release. As demonstrated, release of MTX can be triggered by glutathione. It is known that cancer cells/tissues ubiquitously express higher glutathione levels compared to normal cells/tissues (reviewed by Balendiran et. al., Cell Biochem Funct; 22:343-52 (2004)). High glutathione expressions in AML cell line THP-1 and primary AML cells were confirmed by measuring intracellular glutathione concentrations using an enzymatic recycling assay compared to hematopoietic stem/progenitor cells (HSPCs) from cord blood, a relevant normal cell comparison.

In summary, Au nanoparticles directly passivated with unmodified drugs were evaluated as a drug delivery platform. Au nanoparticles could be practical drug delivery vehicles, since thorough toxicology studies as well as phase I trials indicate minimal or no toxicity at clinically relevant dosages. Murphy et al., Acc Chem Res; 41:1721-30 (2008). Using Au:MTX as an example, the inventors demonstrated the possibility of controlled drug release through competitive affinity between MTX and GSH towards the Au nanoparticle core. Au:MTX exhibited enhanced therapeutic index in the AML models in vivo and in vitro, possibly through the payload release via ligand exchange, although the detailed mechanism requires further elucidation. The simplicity of the synthesis and composition of these drug-Au nanoparticles facilitates scale-up for clinical evaluation, and versatile application to multitude of therapeutic agents in common use. Controlling nanoparticle size and surface properties would affect the bio-distribution and may further enhance the therapeutic index. Libutti et al., Clin Cancer Res 2010; 16:6139-49; De Jong et al., Biomaterials; 29:1912-9 (2008). Post-synthesis partial ligand exchange to incorporate additional functional molecules that enhance targeting, and/or to confer a tracking modality (e.g., fluorescence), is also feasible and is being evaluated.

### Example 2

### Conjugation Using Partial Ligand Exchange

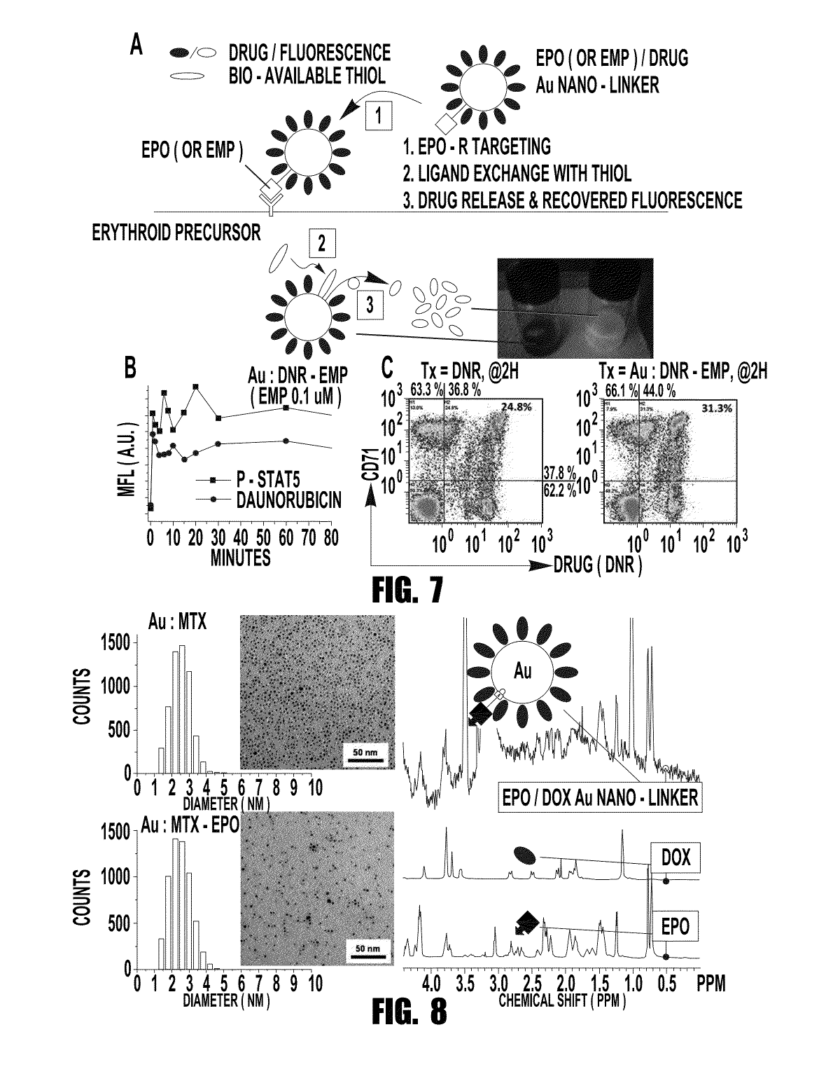

The inventors have obtained conclusive data that targeting moieties can be conjugated to Au nano-linker via simple partial ligand exchange (FIGS. 7, 8). Targeting molecules of a wide variety of sizes are compatible with Au nano-linker synthesis. These include large molecules (˜10-100 kDa) such as recombinant EPO (erythropoietin), recombinant GCSF (granulocyte colony-stimulating factor), mAb (monoclonal antibodies), as well as smaller (˜1 kDa) molecules including peptides such as erythroid progenitor or myeloid targeting peptides (ETPs or MTPs) (FIGS. 7, 8).

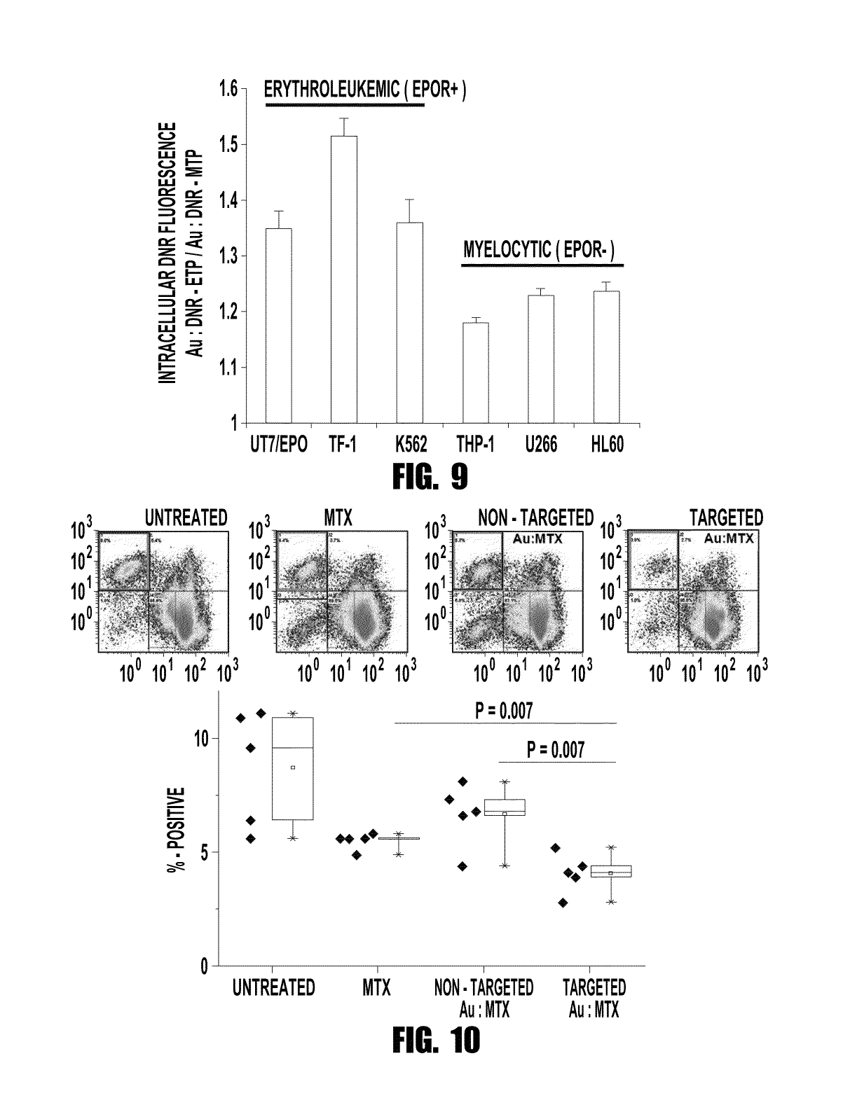

The inventors have demonstrated in vitro that: (a) a multitude of targeting moieties, including recombinant EPO and erythroid progenitor targeting peptides including EPO mimetic peptides, can readily form stable covalent bond to Au nano-linker through partial ligand exchange; (b) cellular uptake as well as drug release, confirmed by fluorescence microscopy and flow cytometry; (c) time-course of the binding to EPO receptor (EPO-R; JAK2/pStat5 pathway activation) and drug release simultaneously using intracellular phospho flow cytometry (FIG. 7). The inventors have also demonstrated in vivo the selective cell-lineage targeting within the same tissue compartment (bone marrow) (FIG. 9).

Synthesis and detailed characterization of drug nano-linked to targeting moieties, to evaluate targeting of erythroid versus myeloid cells by various off-the-shelf drugs conjugated to a range of lineage-targeting molecules. The inventors used drugs including daunorubicin (DNR), doxorubicin (DXR), MTX, or cytarabine monophosphate, and evaluated the linkage of targeting moieties (TMs) including EPO, GCSF, ETP, and MTP, using Au nano-linker. The synthesis is completed in two simple steps, without using complicated chemistry: first, amine-Au nano-cores were synthesized using unmodified drug molecules as ligands; second, simple addition of targeting molecules to the drug-Au nano-core solution readily induced covalent bonding of targeting molecules to Au core, through amine-to-thiol partial ligand exchange.

Drug loading. The synthesis employs a “one-pot” approach: amine-containing molecules and Au precursor are mixed and reacted in one flask. Important parameters in the inventors one-pot synthesis have been identified: molecular weight of the amine-containing molecule as ligands; reaction solvent with different polarity, reducing agent, and temperature.

Targeting moiety (TM) conjugation. The inventors used EPO, GCSF, ETP, or MTP as TMs. Irreversible linkage to Au nano-core surface was induced through thiol-Au interaction at the cysteine residues, naturally included in the TMs or added at the C-terminus of the peptide sequence. Alsina J, Albericio F., Biopolymers; 71:454-77 (2003). Conjugation proceeds simply by mixing these TMs to drug-Au nano-linker solution at desired TM-to-Au molar ratios, in contrast to the multi-step conventional linker chemistry. Leriche et al., Bioorg Med Chem; 20:571-82 (2012). The TM-to-Au molar ratio, as well as the conjugation conditions including solvent, temperature, and pH, were explored. Detailed structural information of the Au nano-linker was assessed using a range of physical chemistry techniques including optical spectroscopy, TEM (transmission electron microscopy), mass spectrometry—specifically MALDI (matrix assisted laser disorption ionization) and ESI (electrospray ionization), elemental analysis (CHN, carbon-hydrogen-nitrogen, analysis combined with ICP-MS, inductively-coupled plasma mass spectrometry), ¹H-NMR (proton nuclear magnetic resonance) spectroscopy, and FTIR (Fourier transform infra-red) spectroscopy.

### Example 3

### In Vitro Demonstration of Controlled Drug Release and Targeting

Cellular uptake of Au nano-linkers and controlled drug release kinetics in vitro. The inventors characterized the glutathione (GSH, ubiquitous endogenous thiol) concentration-dependent controlled drug release kinetics in vitro from Au core using AML drugs. The natural fluorescence of drugs is quenched while they are attached to Au core, but is recovered upon release (FIG. 7). This was exploited to elucidate the drug release process of daunorubicin-loaded Au nano-linker using fluorescence spectroscopy in test tube, as well as flow cytometry and fluorescence microscopy in vitro using harvested murine bone marrow as well as cell lines, such as UT7/EPO, TF-1, K562, THP-1, U266, and HL-60. Evidence of intracellular delivery by Au nano-linkers was confirmed via intracellularly released and recovered drug fluorescence, as well as using transmission electron microscopy (TEM) imaging.

Cellular uptake of Au nano-linkers and controlled drug release kinetics in vitro. The inventors characterized the glutathione (GSH, ubiquitous endogenous thiol) concentration-dependent controlled drug release kinetics in vitro from Au core using AML drugs. The natural fluorescence of drugs is quenched while they are attached to Au core, but is recovered upon release (FIG. 7). This was exploited to elucidate the drug release process of daunorubicin-loaded Au nano-linker with ETP (Au:DNR-ETP) or daunorubicin-loaded Au nano-linker with MTP (Au:DNR-MTP) using fluorescence spectroscopy in test tube, as well as flow cytometry and fluorescence microscopy in vitro using EPO-R expressing cell lines, such as UT7/EPO, TF-1, or K562, and GCSF-R expressing cell lines, such as THP-1, U266, or HL-60. See FIG. 9. These measurements were done with or without the conjugation of TM to the Au nano-linker, to quantitatively elucidate the targeted drug delivery to erythroid versus myeloid lineage. Evidence of intracellular delivery by Au nano-linkers was confirmed using TEM imaging. Lineage selective delivery was demonstrated by measuring fluorescence of intracellularly released DNR using flow cytometry.

In vitro proof-of-efficacy using TM/doxorubicin Au nano-linker in mouse bone marrow. Target selectivity was assessed in vitro by comparing Au nano-linker delivery of fluorescent drug (e.g. daunorubicin, doxorubicin) to a mixture of target and non-target cells. This can be most elegantly demonstrated using whole bone marrow, where myeloid cells coexist with stem/progenitor populations of other lineage (e.g. erythroid stem/progenitor cells). The target selectivity of Au nano-linkers with TM/doxorubicin (TM=EPO, GCSF, ETP, or MTP) was documented in vitro by two-color flow cytometry measuring the cell surface lineage marker and intracellularly released drug fluorescence (FIGS. 6, 7). Moreover, to quantitatively understand the Au nano-linker drug delivery process, multi-color intracellular phospho-flow cytometry was employed, and the timings of events including TM-to-receptor binding (probed by measuring phosphorylated Stat5 in JAK2/Stat5 pathway activation) and intracellular doxorubicin fluorescence that is recovered upon release were interrogated. These studies were conducted along with controls: doxorubicin alone; Au nanoparticle alone; doxorubicin Au nano-linker without the conjugation of targeting moieties; and doxorubicin Au nano-linker with conjugation of scrambled peptide sequence. The simultaneous time-course kinetics of drug delivery and receptor activation were elucidated.

### Example 4

### In Vivo Proof of Principle

The selective targeting of one cell population over another was evaluated within the same tissue compartment in vivo. To demonstrate the proof of principle, erythroid lineage-targeting Au nano-linkers with MTX payload, versus the set of controls—Au:MTX with scrambled peptide, Au:MTX, MTX alone, Au nanoparticle alone, and vehicle treatment in C57/BL6 mice (n=5 per treatment group). The compounds were administered intravenously (i.v.) 3×/week for 2 weeks, and mice were sacrificed for end-point evaluation. The end-point is murine CD71+/CD45-erythroid lineage cells versus non-erythroid lineage cells. This primary end-point was compared between groups with Wilcoxon test of bone marrow flow cytometric analysis, bone marrow Wright-Giemsa stains, blood counts, and blood smears (FIG. 10).

The complete disclosure of all patents, patent applications, and publications, and electronically available materials cited herein are incorporated by reference. The foregoing detailed description and examples have been given for clarity of understanding only. No unnecessary limitations are to be understood therefrom. In particular, the inventors are not bound by theories described herein. The invention is not limited to the exact details shown and described, for variations obvious to one skilled in the art will be included within the invention defined by the claims.

What is claimed is:

1. An amine-passivated gold nanoparticle, comprising a gold nanoparticle passivated with a plurality of amine-containing drugs or imaging agents.

2. The nanoparticle of claim 1, wherein the nanoparticle has a diameter from 1 to 5 nanometers.

3. The nanoparticle of claim 1, wherein the amine-containing drugs or imaging agents have a molecular weight from about 300 to about 1,000 daltons.

4. The nanoparticle of claim 1, wherein the nanoparticle is passivated with an amine-containing drug.

5. The nanoparticle of claim 1, wherein the amine-containing drug is selected from the group consisting of guanosine, cytidine monophosphate, guanosine monophosphate, cytidine diphosphate, thyamine pyrophosphate, adenosine diphosphate, folic acid, guanosine diphosphate, tetracycline, oxytetracycline, doxycycline, methotrexate, methotrexate dimethyl ester, cytidine triphosphate, adenosine triphosphate, guanosine triphosphate, daunorubicin, idarubicin, doxorubicin, streptomycin, and flavin adenine dinucleotide.

6. The nanoparticle of claim 1, wherein the nanoparticle is passivated with an amine-containing imaging agent.

7. The nanoparticle of claim 1, wherein the amine-passivated gold nanoparticle further comprises a targeting molecule bonded to the nanoparticle through a thiol linkage.

8. The nanoparticle of claim 7, wherein the targeting molecule is selected from the group consisting of granulocyte colony stimulating factor (GCSF), a GCSF mimetic peptide, erythropoietin, erythropoietin mimetic peptide, and a myeloid targeting cell penetrating peptide.

9. A method of making an amine-passivated gold nanoparticle, comprising combining an amine-containing drug or imaging agent with HAuCl₄ and a reducing agent in a polar organic solvent at a temperature from −80 to 20° C. for a time sufficient to form amine-passivated gold nanoparticles.

10. The method of claim 9, wherein the gold nanoparticle has a diameter from 1 to 5 nanometers.

11. The method of claim 9, further comprising reacting the amine-passivated gold nanoparticle with a thiol-containing targeting molecule.

12. The method of claim 9, wherein the reducing agent is sodium borohydride.

13. A method of treating cancer in a subject identified as having cancer by administering to the subject a therapeutically effective amount of an amine-passivated gold nanoparticle comprising a gold nanoparticle passivated with an amine-containing anticancer agent.

14. The method of claim 13, wherein the amine-containing compounds have a molecular weight from about 300 to about 1,000 daltons.

15. The method of claim 13, wherein the cancer is acute myeloid leukemia.

16. The method of claim 13, wherein the amine-passivated gold nanoparticle has a diameter from 1 to 5 nanometers.

17. The method of claim 13, wherein the amine-passivated nanoparticle is administered together with a pharmaceutically acceptable carrier.

18. The method of claim 13, wherein the amine-passivated nanoparticle further comprises a targeting molecule bonded to the nanoparticle through a thiol linkage.

19. The method of claim 18, wherein the targeting molecule is selected from the group consisting of granulocyte colony stimulating factor (GCSF), a GCSF mimetic peptide, erythropoietin, erythropoietin mimetic peptide, and a myeloid targeting cell penetrating peptide.

20. The method of claim 13, wherein the amine-containing anticancer agent is selected from the group consisting of methotrexate, methotrexate dimethyl ester, daunorubicin, idarubicin, and doxorubicin.
